(19) United States
(12) Patent Application Publication
(10) Pub. No.: US 2026/0283156 A1
(43) Pub. Date: Sep. 24, 2026

(54) LARGE VOLUME CHLORINE DIOXIDE STERILIZATION AND/OR DISINFECTION SYSTEM AND METHOD OF USING SAME

(71) Applicant: STERIGENICS NV, Wijgmaal (BE)

(72) Inventors: Aaron DEMENT, Schaumburg, IL (US); Erik HAGHEDOOREN, Wijgmaal (BE); Gregory GRAMS, Wijgmaal (BE); Eric OFFERMANN, Wijgmaal (BE); Marie-Pierre HILDEBRAND, Wijgmaal (BE)

(21) Appl. No.: 19/478,304

(22) PCT Filed: Apr. 26, 2024
(86) PCT No.: PCT/US2024/026524

§ 371 Date: Oct. 24, 2025

(30) Foreign Application Priority Data
Apr. 26, 2023 (US) 63461946

(51) Int. Cl. A01N 59/00; A01P 1/00; A61L 2/20; A61L 2/24; A61L 101/06; C25B 1/26

(52) U.S. Cl. CPC A01N 59/00; A01P 1/00; A61L 2/20; A61L 2/24; A61L 2101/06; A61L 2202/11; A61L 2202/122; A61L 2202/13; A61L 2202/14; C25B 1/26

(57) ABSTRACT

A decontamination method including the steps of arranging an article within a space area, introducing chlorine dioxide gas from an associated system into the space area, sterilizing the article, and venting the chlorine dioxide into a stream or volume of water.

### BACKGROUND OF THE INVENTION

The present invention relates to an apparatus and method for sterilization and/or disinfection by exposure to an atmosphere having an effective amount of chlorine dioxide gas in a treatment chamber to reduce microorganisms. The present application claims the benefit of U.S. Provisional Application No. 63/461,946 filed Apr. 26, 2023, the disclosure of which is herein incorporated by reference.

### DESCRIPTION OF RELATED ART

Chlorine dioxide (CD or ClO₂) was discovered in the early 1800's and has been approved for a wide variety of commercial disinfecting/sterilizing applications by the EPA, FDA and USDA. Due to its demonstrated efficacy with respect to a wide variety of contaminated surfaces, ClO₂ has been called the ideal biocide and the ability of chlorine dioxide to reduce or eliminate microbes, e.g., bacteria, viruses, fungi, mold spores, algae, and protozoa, at relatively low concentrations is well-documented. Because ClO₂ inactivates microorganisms by oxidizing critical components of a microorganism's membrane proteins, tolerance to ClO₂ does not develop, making it an ideal disinfectant/sterilant for repeated-use applications.

ClO₂ is a green-yellowish gas with a chlorine-like odor; however, ClO₂ is a neutral chlorine compound. ClO₂ is a small, volatile, and very strong molecule. In diluted, aqueous solutions ClO₂ is a free radical. At high concentrations it reacts strongly with reducing agents. Chlorine dioxide is an unstable gas that dissociates into chlorine gas and oxygen gas readily. Further, ClO₂ may be photo-oxidized by sunlight and therefore decontamination applications generally proceed in the absence of light. The end-products of ClO₂ neutralization/degradation reactions are chloride (Cl⁻), chlorite (ClO₂⁻) and chlorate (ClO₃⁻).

A significant drawback of ClO₂ is that it is explosive under pressure, making transport in bulk (e.g. tanker) a challenging proposition. Hence it is typically manufactured on site (in situ). ClO₂ is usually produced as an aqueous solution or gas. It is produced in acidic solutions of sodium chlorite (NaClO₂), or sodium chlorate (NaClO₃). Sodium chlorite, chlorine gas (Cl₂), sodium hydrogen chlorite (NaHClO₂) and sulphuric or hydrogen acid are typically used to produce chlorine dioxide on site.

Decontamination systems which exploit the beneficial properties of ClO₂ fumigant are known in the art. However, these systems generally suffer from production of excess humidity with the fumigant, resulting in production of hydrochloric acid mist and potential to corrode electronic equipment, making the system inconvenient for large-scale building decontamination, since removal of corrosion-sensitive articles must be effectuated prior to decontamination.

U.S. Pat. No. 8,524,167 (the '167 patent) discloses a ClO₂ decontamination system, however it suffers from a failure to provide mechanisms for removal of byproducts and relies on humidification as a necessary aspect of effective ClO₂ fumigant decontamination. Moreover, chlorine gas is produced because of humidification. Accordingly, The '167 patent system is unsuitable for corrosion-sensitive articles and environments. Further, the '167 fumigant scrubber relies heavily on carbon, which is rendered less effective by the presence of water. Notably, the use of carbon filtration with non-degraded ClO₂ can create an explosive potential because ClO₂ can build up in the carbon pores in problematic concentrations. Hence, the use of carbon as a primary neutralizer/scrubber presents a fire and safety hazard.

U.S. Pat. App. Pub. No. US 2012/0164025A1 discloses a package and method for disinfecting microbiologically contaminated products using a ClO₂ gas generating sachet. A predetermined volume of reactants for generating and neutralizing the ClO₂ disinfectant gas are pre-sealed within the gas generating sachet and separated by a frangible seal. A product to be disinfected is inserted into a package with the sachet and the package is sealed. The gas generation sachet is then physically activated by manually breaking the frangible seal. After a period of time, the package is unsealed, and the product removed. In addition to the other shortcomings related to sachet-based gas generation systems noted above, pre-packaged gas generation sachets and disinfection methods employing them suffer from a lack of flexibility and control over the disinfection process. Both the length of the process and the concentration of disinfection gas are predetermined by the volume and amount of the reactants pre-sealed in the gas generating sachet. Thus, both the required concentration of disinfecting gas and the required disinfection time must be estimated in advance. Because it is not possible to terminate or otherwise control or adjust the disinfection process once it is underway, if the advance estimates are wrong, more disinfection than necessary may take place with an attendant waste of time, money, and chemicals, or insufficient disinfection may take place and require additional disinfection cycles.

U.S. Pat. No. 9,943,620 discloses a portable chlorine dioxide decontamination system. The disclosure is particularly focused on the use of a fumigant activating area where an activation cup of chlorine dioxide reagents are provided. This disclosure relies on an inefficient mechanism for generation of ClO2. In addition, this disclosure requires zeolite treatment and charcoal filtration of the ClO2 after sterilization. Accordingly, the apparatus of this disclosure is incapable of treating large volumes of articles for sterilization with chlorine dioxide.

Clearly there remains a need in the art for safe and effective ClO₂ fumigant decontamination system that minimizes the use of water, minimizes agitation/degradation of the CD fumigant, and avoids dispersal of water vapor, acid and chlorine gas along with the fumigant. In addition, there remains a need for a system that provides adjustability of the decontamination process driving an active decontamination cycle. Furthermore, the above-described decontamination systems only provide for treatment on a very small scale. Accordingly, there remains a need for an apparatus capable of providing ClO₂ decontamination on a bulk scale.

### SUMMARY OF THE INVENTION

Various details of the present disclosure are hereinafter summarized to provide a basic understanding. This summary is not an extensive overview of the disclosure and is neither intended to identify certain elements of the disclosure, nor to delineate scope thereof. Rather, the primary purpose of this summary is to present some concepts of the disclosure in a simplified form prior to the more detailed description that is presented hereinafter.

The present disclosure provides an apparatus and method for reducing microorganisms by treating an article in a large volume treatment chamber with an atmosphere having an effective amount of chlorine dioxide.

According to one embodiment, the sterilization apparatus is configured for reducing microbial content of an article. The apparatus includes a chlorine dioxide generation system for generating chlorine dioxide gas. The apparatus includes a treatment chamber configured to receive the article, the treatment chamber being pressurized between 2 and 120 kPa, with an atmosphere that can have a relative humidity from about 20% to about 100% at a temperature from about 2 degrees Celsius to about 80 degrees Celsius. The apparatus further includes a treatment station for dissolving the used chlorine dioxide gas in water and/or air. The apparatus will include one or more sensing unit(s) in combination with a controller having software configured to sense and optionally adjust one or more conditions in the chlorine dioxide generation reactor, treatment chamber, and/or water treatment station.

According to another embodiment, a sterilization apparatus configured for reducing microbial content of an article is provided. The apparatus includes a chlorine dioxide generation system for generating chlorine dioxide gas according to a scheme in which 2NaClO2+2H₂O→2ClO₂+H₂+2NaOH. The scheme can optionally be based upon an anode oxidation reaction (NaClO₂→ClO₂+e—) and a cathode reduction reaction (2H₂O+2e—→H₂+₂OH—).

According to an additional embodiment, the sterilization apparatus is configured for reducing microbial content of an article. The apparatus includes (i) a chlorine dioxide generation system for generating chlorine dioxide gas from a reaction between chlorite and an acid, (ii) an optional buffer tank receiving the chlorine gas and building a pressure of at least 100 kPa, (iii) a treatment chamber configured to receive the article, said treatment chamber being pressurized between 2 and 120 kPa, (iv) an optional evacuation tank for receiving the chlorine dioxide gas after exposure of the article, optionally including a degradation enhancement device, (v) an optional ClO₂ scrubber to reduce the chlorine dioxide gas, (vi) a water treatment station for dissolving the used chlorine dioxide gas from step (iv) in water and/or air, and (vii) one or more sensing unit(s) in combination with a controller having software configured to sense and optionally adjust one or more conditions in the chlorine dioxide generation reactor, buffer tank, treatment chamber, evacuation tank, ClO₂ scrubber and/or water treatment station.

According to a further embodiment, a sterilization method is provided which includes the steps of arranging an article within a space area, introducing chlorine dioxide gas from an associated reactor into the space area, sterilizing/disinfecting the article, and venting the chlorine dioxide into a stream or volume of water or air or both.

The method of the present disclosure is advantageous relative to the use of ethylene oxide which is a carcinogen.

### DESCRIPTION OF THE DRAWINGS

The following is a brief description of the drawings, which are presented for the purposes of illustrating the exemplary embodiments disclosed herein and not for the purposes of limiting the same.

FIG. 1 is a schematic illustration of a sterilization apparatus of the present disclosure;

FIG. 2 is a schematic illustration of an alternative sterilization apparatus of the present disclosure; and

FIG. 3 is a schematic illustration of an exemplary chlorine dioxide generation reactor.

### DETAILED DESCRIPTION

A more complete understanding of the components, processes, and apparatuses disclosed herein can be obtained by reference to the accompanying drawings, these figures are merely schematic representations based on convenience and the ease of demonstrating the present discloser, and are, therefore, not intended to indicate relative size and dimensions of the devices or components thereof and/or to define or limit the scope of the exemplary embodiments.

Although specific terms are used tin the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a”, “an”, and “the” include plural referents unless the context clearly dictates otherwise.

As used herein, the terms about, generally, and substantially are intended to encompass structure or numerical modifications which do not significantly affect the purpose of the element or number modified by such term.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such descriptions should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

Gas sterilization is known for the disinfecting treatment of unsterilized articles. As an example, a gas sterilizer is used to first feed a sterilizing gas from a gas generator or gas tank into a sterilization chamber and then to keep the chamber in the gas exposure condition for a certain period of time to annihilate microorganisms and bacteria floating in the air or adhering on the article being sterilized. As such gas sterilization can be used to sterilize microbially contaminated medical devices for their recycle or initial use.

Sterilization of medical instrument often utilizes autoclaves, however, they disadvantageously require heating to a temperature of 130 degrees C. Accordingly, autoclaves cannot be used with low heat resistant instruments, for example, those made of rubber or pharmaceutical materials that may degrade or food stuff, as examples.

In some embodiments, the present disclosure is directed to chlorine dioxide sterilization which is recognized to have certain benefits with respect to articles suitable for sterilization that are not effectively treated with other gasses such as ozone, and nitrogen oxide. The present disclosure is also more environmentally friendly than sterilization with ethylene oxide.

The term “sterilization” herein means an aseptic condition of all proliferative microorganisms (mainly bacteria) being completely removed and destroyed or a condition of the possibility of microorganism growth being essentially close to zero, i.e., the condition of the sterility assurance level (SAL) less than 10⁻⁶ in the existing probability of microorganisms after sterilization, based on a method-validation for the particular article. In a sterilization process, the nature of microbial inactivation is exponential and thus the survival of a microorganism on an individual item can be expressed in terms of probability. While this probability can be reduced to a very low number, it can never be reduced to zero.

Disinfection and sterilization are both decontamination processes. While disinfection is the process of eliminating or reducing harmful microorganisms from inanimate articles and surfaces, sterilization is the process of killing all microorganisms, including spores of various organisms present on surfaces. Sterilization requires a validated method for the article to demonstrate a defined SAL.

It is noted that the present disclosure is intended to cover a process, a system and an apparatus that is suitable for each of sterilization, disinfection and decontamination and those terms may be used interchangeably herein.

An illustrative embodiment of this invention comprises an article receiving treatment chamber, a means for providing a gas stream comprising chlorine dioxide (optionally with an inert carrier gas such as nitrogen) into the treatment chamber, a chlorine dioxide sensor in communication with the treatment chamber, a means for controlling the concentration of chlorine dioxide in the treatment chamber in response to the sensor, and a means for removing the chlorine dioxide from the treatment chamber for introduction into a volume of water and/or air.

Advantageously, the treatment chamber includes a mechanism for controlling temperature. This alone and in combination with pressure and humidity controls allows the present system to decontaminate a very wide array of articles. Furthermore, the ability to control both temperature and pressure provides a safe environment for performing ClO₂ decontamination. These advantages are achieved by providing the system with sensors in association with many if not all components. Such sensors are linked with a controller to allow the system to operate in an intelligent manner. Furthermore, the sensors/controller allow the system to modify a decontamination sequence during processing. For example, the system could monitor the process via sensing of SAL or other condition determined important for validation.

It is noted that the phrases “ClO₂ generation delivery system” and “chlorine dioxide generation reactor” may be used intermittently herein. However, these terms are intended to encompass mechanisms that generate ClO₂ in any manner consistent with the intentions of this disclosure as understood by the skilled artisan.

In an illustrative embodiment of the invention, the treatment chamber is an elongated chamber suitable to receive at least one pallet of articles to be treated, a transportation container, a truck, or a conveyor upon which articles to be treated are moved.

Of course, the disclosure is not intended to be limited in all embodiments to large volume sterilization scenarios. Rather, select configurations of the apparatus components are anticipated to have application with a smaller scale of articles being treated, e.g., 4200 liter volume, or even laboratory size applications.

The means for providing a gas stream can comprise a generator having a source of chlorite for generating a gas stream comprising of chlorine dioxide gas and optionally a carrier gas. The generated gas stream flows into the treatment chamber to form the atmosphere inside the treatment chamber for exposing the article thereto with or without a buffer tank.

The means for controlling the concentration of chlorine dioxide can comprise a feedback control system connected to the chlorine dioxide sensor. Concentration of chlorine dioxide could be measured via mass, pressure or directly. In this context, the sensor may be associated with one or more of the outputs from the generator, the buffer tank, the treatment chamber, the evacuation tank, or other location beneficial in controlling the sterilization process.

The feedback control system can be a logic controller that is pre-programmed to take certain action upon receiving set parameters. For example, the buffer tank may be emptied into the treatment chamber when a suitable chlorine dioxide pressure is reported by the sensor to the PLC. As a further example, the time for a pre-set sterilization cycle can be counted down by the PLC when a suitable chlorine dioxide concentration is reported by a sensor in the treatment chamber. Similarly, article(s) may be emptied from the evacuation chamber when a suitably low chlorine dioxide level is detected by a sensor associate therewith as determined by the PLC.

A first advantage of the present disclosure is to provide a method and an apparatus for safely sterilizing/decontaminating microbially contaminated articles with chlorine dioxide. A second advantage of the present disclosure is to provide a method and an apparatus for rapidly and certainly sterilizing complexly configured articles. A third advantage of the present disclosure is to provide a method and an apparatus for effectively recovering a sterilant used for sterilization without any remaining harmful sterilant on sterile articles. A fourth advantage of the present disclosure is to provide an apparatus and a method for safely sterilizing airborne microorganisms and nonsterile articles within a space area typically at an atmospheric pressure. A fifth advantage of the present disclosure is to provide a safely and easily handled apparatus and method for sterilization by means of chlorine dioxide as a sterilant feedstock. A sixth advantage of the present disclosure is to provide an apparatus and a method for rapidly generating a sterilant at a predetermined concentration to reliably sterilize microorganisms and unsterilized articles within a space area. A seventh advantage of the present disclosure is to provide an inexpensive apparatus and method of treating large volumes of articles simultaneously (e.g., multiple pallets).

Example embodiments are directed to a chlorine dioxide decontamination system and method. The disclosed chlorine dioxide generation system provides a ClO₂ fumigant that is optionally substantially free of water vapor, acid vapors and other by-products of ClO₂ gas production. Thus, the ClO₂ decontamination system may be utilized in a broad range of applications, including for example, sterilization of corrosion-sensitive electronic equipment and metallic substrates, sterilization of medical articles, pharmaceutical products, cannabis, food, and sterilization of devices that may be subject to repeated sterilization.

Further, the ClO₂ fumigant decontamination system provides an aspect of one-pass neutralization, simplifying and decreasing the expense and time associated with large-scale decontamination projects generally. The system also provides flexibility and control over the decontamination process while it is underway. Still further, the system is adapted to provide not only decontamination of large articles, but also sterilization of bundled small articles, including articles enclosed in sealed packages and the openings and internal channels and spaces within such articles.

### Embodiment 1

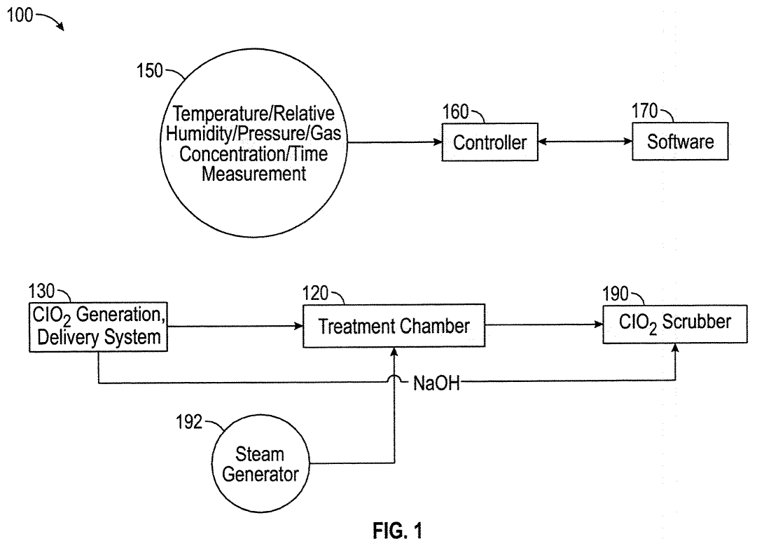

Referring to FIG. 1, an embodiment of the disclosure provides an apparatus 100 for exposing articles to an atmosphere comprising gaseous chlorine dioxide and optionally a carrier gas in a treatment chamber 120.

The apparatus can include a system 130 for generating and delivering a gas stream of ClO₂ and optionally a carrier gas. This is called the gas mixture. The gas mixture can be transferred to the treatment chamber 120 at a desired pressure, for example of at least 100 kPa.

In one embodiment it may be desirable that the treatment chamber is operated at ambient pressure.

Chamber 120 can be configured to contain articles for which a reduction in microorganisms thereon/therein is desired. The chamber may optionally receive steam from steam generator 192.

After sufficient sterilization of the articles in the treatment chamber 120 has been completed, which could be determined by a preset cycle time or by sensing microbe concentration, as examples, the remaining gas mixture can be evacuated to a scrubber 190 where the ClO₂ gas can be reduced before discharge to air and/or water.

The gas mixture stream can optionally be transferred from the scrubber 190 to a water treatment station 194 (needs to be readded to Fig.) for dissolving the chlorine dioxide gas.

The treatment chamber 120 can also be configured to recycle ClO₂ gas to the generation system 130.

The parameters of temperature, relative humidity, pressure, gas concentration and time can be measured using various sensors 150 associated with the ClO₂ generation system 130, treatment chamber 120, ClO₂ scrubber 190, steam generator 192 and/or an optional water treatment station and communicated to a controller 160.

Data captured by the various sensors in the different components of the system can be transferred to the controller 160 where software 170 is employed to control the system, ensuring sterilization/decontamination cycles with the correct parameters of temperature, relative humidity, pressure, gas concentration and time to ensure microorganism reduction on articles. The parameters of temperature, relative humidity, pressure, gas concentration and time can be measured using various sensors 150 connected with the ClO₂ generation, delivery system 130, treatment chamber 120, ClO₂ scrubber 190, steam generator 192 and/or the optional water treatment station and communicated to a controller 160. Lines are not shown between each of these potential connections to improve the clarity of the image and/or because such communication can be wireless.

### Embodiment 2

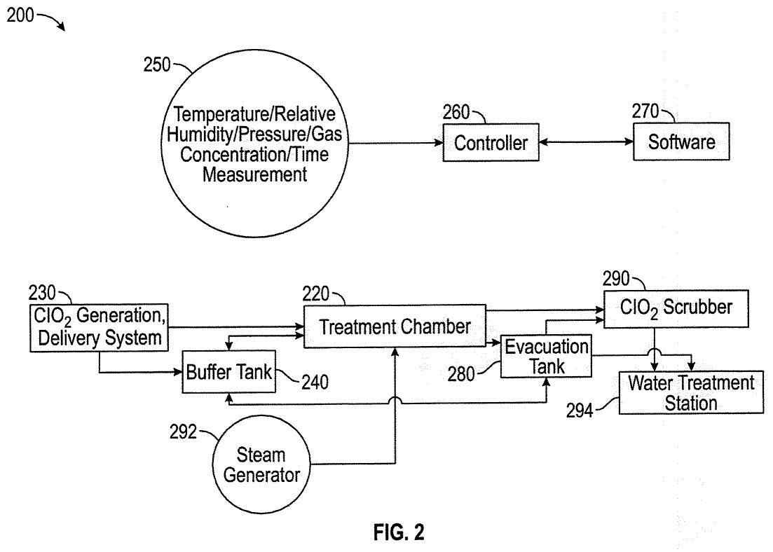

Referring to FIG. 2, an embodiment of the disclosure provides an apparatus 200 for exposing articles to an atmosphere comprising gaseous chlorine dioxide and optionally a carrier gas in a treatment chamber 220.

The apparatus can include a system 230 for generating and delivering a gas stream of ClO₂ and optionally a carrier gas. This is called the gas mixture. The gas mixture can be transferred to a buffer tank 240 that will build a pressure, for example of at least 100 kPa, to ensure a pressure differential between the system 230 and the treatment chamber 220. In some embodiments the buffer tank 240 will be regulated and monitored by at least one sensor to ensure control of the gas mixture stream concentration.

The gas mixture stream can be transferred from the buffer tank 240 to the treatment chamber 220 with a circulation blower, for example, to ensure homogenous conditions. The disclosure also contemplates an embodiment where the treatment chamber 220 receives the gas mixture directly from the system for generating and delivering a gas stream 230. In one embodiment gas flow is directed from the system for generating and delivering a gas stream 230 to the treatment chamber, it may be desirable that the treatment chamber is operated at ambient pressure.

The chamber can be configured to contain articles for which a reduction in microorganisms thereon/therein is desired. The chamber may optionally receive steam from a steam generator 292.

After sufficient sterilization of the articles in the treatment chamber 220 has been completed, which could be determined by a preset cycle time or by sensing microbe concentration, as examples, the remaining gas mixture can be transferred to an evacuation tank 280.

The evacuation tank 280 may include a ClO₂ a gas degradation enhancement device. For example, UV light exposure could be provided.

The gas mixture stream can be transferred from the treatment chamber 220 and/or the evacuation tank 280 to a scrubber 290 where the ClO₂ gas can be reduced before discharge to air and/or water.

The gas mixture stream can be transferred from the scrubber 290 and/or evacuation tank 280 to a water treatment station for dissolving the chlorine dioxide gas received 294.

The evacuation tank 280 and/or treatment chamber 220 can also be configured to recycle ClO₂ gas to the buffer tank 240.

The parameters of temperature, relative humidity, pressure, gas concentration and time can be measured using various sensors 250 associated with the ClO₂ generation system 230, the buffer tank 240, treatment chamber 220, evacuation tank 280, ClO₂ scrubber 290, steam generator 292 and/or water treatment station 294 and communicated to a controller 260.

Data captured by the various sensors in the different components of the system can be transferred to the controller 260 where software 270 is employed to control the system, ensuring sterilization/decontamination cycles with the correct parameters of temperature, relative humidity, pressure, gas concentration and time to ensure microorganism reduction on articles. The parameters of temperature, relative humidity, pressure, gas concentration and time can be measured using various sensors 250 connected with the ClO₂ generation, delivery system 230, the buffer tank 240, treatment chamber 220, evacuation tank 280, ClO₂ scrubber 290, steam generator 292 and/or water treatment station 294 and communicated to a controller 260. Lines are not shown between each of these potential connections to improve the clarity of the image and/or because the communication can be wireless.

The following paragraphs are directed to elements of the decontamination apparatus that are applicable to all embodiments.

The mechanism for providing the gas stream can be commercially available equipment such as the chlorine dioxide generator manufactured by Dioxide Pacific of Sydney Australia for use in potable water disinfection systems and paper whitening processes.

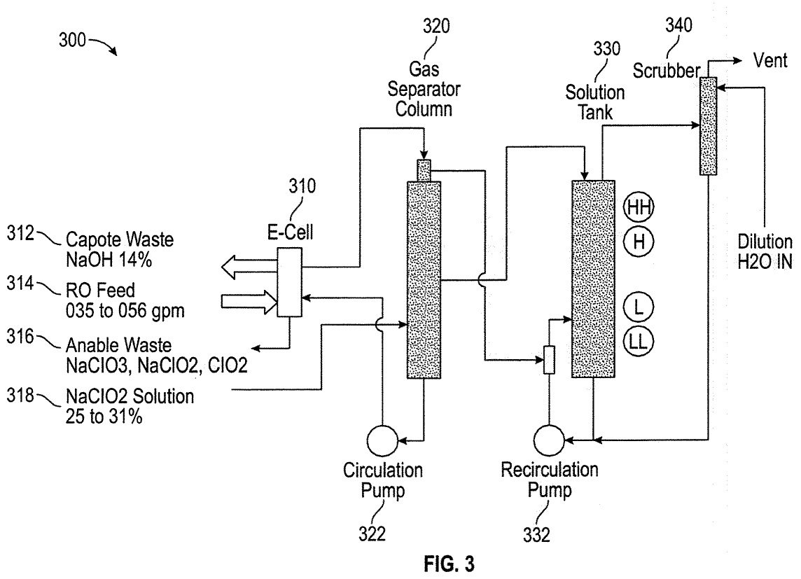

An exemplary system 300 is displayed in FIG. 3. The system can operate using only one precursor chemical and electrical energy to create chlorine dioxide.

The generator includes an electrochemical cell 310 which has an anode (+) side which is supplied with the electricide NaClO₂ solution (e.g. 25 to 31%) from circulation pump 322. Cathode (−) side is supplied with reverse osmosis water 314. Electrical current is applied to the cell electrodes where oxidation and reduction reactions occur. During the chemical reaction in the cells, the Na+ ions pass from the anode side to the cathode side, thus reduction creates sodium hydroxide (also called caustic soda NaOH) and hydrogen at the cathode. Oxidation at the anode electrode converts chlorite ion (ClO₂⁻) to chlorine dioxide (ClO₂).

The mixture coming from cathode side of the cells can be recovered in the scrubber column 340. NaOH can be extracted from the column and recovered in a waste tank (Capote waste NAOH 14%). Anable waste is removed from electrochemical cell 310 via outlet 316 and recovered in a waste tank.

Chlorine dioxide from electrochemical cell 310 and sodium chlorate from feed 318 can be provided to gas separation column 320. The solution tank 330 can be incorporated in the gas separator column 320.

The symbols HH (High High level) and LL (Low low level) represent alarm levels in the solution tank. H and L are the high and low levels of the NaClO₂ solution stored in the solution tank. The tank can be filled with NaClO₂ until reaching the high level, then the filling stops and the solution is used in a decontamination process. Once the low level reached, filling starts again until the high level is reached.

The sodium chlorite can be cooled to 35° C. in a heat exchanger before being sent to the anode of the E-cell. Chlorine dioxide produced in the recirculating anolyte loop of the electrochemical generator can be extracted using a gas separator column and air.

For dosing into water systems, injection of chlorine dioxide gas utilizes a booster ejector. The chlorine dioxide formed can be transferred to the solution tank and dissolved in water. Sodium chlorite can be circulated from separation column 320 via circulation pump 322 to electrochemical cell 310.

For gas phase reactions, chlorine dioxide can be taken from the gas separator column to a mixing point.

Pure chlorine dioxide gas in a stable air mix can be extracted from the gas separator and dissolved into water in a solution tank for example at nominally 2,000 ppm.

In the scrubber column 340, maximum 0.5% v/v H₂ can be diluted with air and evacuated through the vent line to the atmosphere.

Additional details can be derived from www.dioxide.com, the disclosures therein being incorporated herein by reference. In some embodiments, the chlorine dioxide generation system is electrochemical and has a capacity from at least 0.45 kg/day or from at least 136 kg/day ClO₂.

In select embodiments, a vacuum mechanism can be induced to improve emptying the treatment chamber after sterilization of the article. The gas stream generated by the system can either be directly fed into the treatment chamber or diluted by an optional mixing device or pre-staged in a buffer tank. The gas stream comprising chlorine dioxide and optional carrier gas can be circulated inside the chamber by a gas stream circulating device, such as a fan, blower, or any other suitable device for circulating the gas stream. The concentration of chlorine dioxide in the treatment chamber atmosphere can be between about 0.1 to about 100 mg/L, or between about 0.1 to about 10 mg/L, or between about 1 to about 3 mg/L in the treatment chamber atmosphere but can be in an amount sufficient to achieve the benefits or advantages of this invention as described in the examples listed below. During processing, the gas stream can continuously flow into the chamber to maintain a constant concentration of chlorine dioxide or may be pulsed into the chamber.

The chlorine dioxide concentration can be controlled by a feedback control system that includes a control computer connected to the chlorine dioxide sensor and the chlorine dioxide generation system and/or buffer tank. The control computer can control the concentration of chlorine dioxide in the treatment chamber in response to a signal from the chlorine dioxide sensor by controlling the generator or releasing additional chlorine dioxide from the buffer tank.

In certain embodiments, the treatment chamber further can include a magnetic field generator to improve sterilization effectiveness.

Moisture and heat can serve important respective functions in the sterilization process. Moisture can allow for the sterilizing gas to work more effectively. Heat can allow for sufficient sterilization to occur while the articles are exposed to peak sterilizing gas concentrations.

Accordingly, in certain embodiments it may prove beneficial to provide a pre-conditioning chamber or step within the existing treatment chamber for heating and introduction of moisture into the articles being treated.

A steam generator can be provided for humidifying the atmosphere during processing. During treatment, the atmosphere can have a relative humidity from about 20% to about 100% at a temperature from about 2 degrees Celsius to about 80 degrees Celsius. The steam generator can be in communication with the treatment chamber and can maintain a preselected relative humidity within the treatment chamber when the gas stream is present. A feedback control system can be used to control the relative relative humidity in the treatment chamber where a computer controls the steam generator in response to a signal from a relative humidity sensor.

The treatment chamber, the buffer tank and/or evacuation tank can also include a mechanism for increasing the temperature of the ClO₂.

In certain embodiments, the treatment chamber can include a means for aeration after sterilization of the articles.

The chlorine dioxide solution can be removed from the treatment chamber using a purging means or a vacuum. The purged chlorine dioxide can be recycled to the buffer tank or partially neutralized. Purging gasses could be e.g. nitrogen or air.

A chlorine dioxide neutralizer device can be a scrubber that neutralizes the chlorine dioxide by spraying a reducing agent, such as sodium sulfite, sodium thiosulfate or sodium hydroxide. In certain embodiments, the evacuation chamber includes a means for aeration. Alternatively, the neutralizing can be performed with a UV lamp and/or a microwave generator. The residue of chlorine dioxide in the air released from the chlorine dioxide neutralizing device is preferable lower than 0.1 ppm by volume.

The residual chlorine dioxide gas can be introduced to a volume of water, preferably at a concentration that allows for discharge into an existing storm sewer, retention basin or other body of water.

Traditionally, the chamber sterilization/disinfection process can include at least three phases: (i) preconditioning, (ii) sterilization, and (iii) degassing. In the preconditioning phase, the articles to be sterilized/disinfected are already sealed in their final packaging. The articles are first palletized and then placed in a preconditioning room. The temperature and the relative humidity in this preconditioning room are set generally between about from about 2 degrees Celsius to about 80 degrees Celsius and between about 20 and about 95 percent relative humidity, respectively. These conditions are maintained throughout the preconditioning phase according to a validated time frame. Preconditioning and degassing are optional.

Articles contemplated for sterilization and/or decontamination using the present apparatus/process include the non-limiting examples of prefilled syringes, prefilled inhalants, pharmaceutical substances, electronic implants, medical devices, energy storage devices, light density wound dressing, amorphous polymers, stainless steel, galvanized metals, anodized aluminum, painted metals, food and cannabis.

The sterilization phase generally involves transferring the palletized preconditioned articles from the preconditioning room to the sterilization chamber. The size of the sterilization chamber may be configured to receive articles (in total) from about 200 liters in volume to truck sizes of 33 pallets, for example, or more.

The sterilizing chlorine dioxide gas can be introduced into the sterilization chamber. Following the introduction of the sterilizing gas, the pressure level inside the chamber may range from about millibars to about 2 kPa to about 120 kPa. The concentration of chlorine oxide within the chamber can be generally at least 0.1 milligrams per liter (mg/l) and may be as high as 100 mg/l or higher. The duration of exposure to chlorine dioxide may be from about 1 minute to 24 hours or longer.

The degassing phase can follow the sterilization phase. Degassing generally involves moving the sterilized, palletized products from the sterilization chamber to a degassing or aeration room. The temperature in the degassing room is generally maintained between 2 degrees C and 80 degrees C.

The invention is also useful in processing food articles such as fruits and vegetables but is not limited thereto as other kinds of produce such as tubers or other edible plant material can be processed by the present invention for microorganism reduction thereon. In a preferred embodiment, the practice of the invention can achieve at least a 3-log reduction of microorganisms on the produce including but not limited to microorganisms on the exterior surface, cracks, or in other surface irregularities. Microorganisms include any organisms of microscopic or submicroscopic size. In a more preferred embodiment, a 3-log reduction of pathogenic microorganisms present on the produce is attained. Pathogens can include disease-causing microorganisms such as bacteria, viruses, fungi, or spores, including but not limited to enterotoxigenic Escherichia coli 0157:H7, Listeria monocytogenes, Salmonella spp., Shigella spp., Cyclospora, and hepatitis A.

Gaseous chlorine dioxide (ClO₂) treatments can also be an alternative antimicrobial for the elimination of pathogens on fruits, nuts, spices, and vegetables. ClO₂ in both gaseous and aqueous phase is a strong oxidizing and sanitizing agent that has broad and high biocidal effectiveness. It has been reported to effectively inactivate bacteria, including pathogens, virus, bacterial spores, and algae. It has about 2.5 times the oxidation capacity of chlorine. ClO₂ maintains its bactericidal activity far longer than chlorine. It is also less reactive than chlorine with organic compounds, and its use is preferred where high organic loads are encountered. Advantages of ClO₂ over chlorine also include lack of odor and taste, effectiveness at low concentration, non-conversion to chlorophenols which result in residual smells and flavors, ability to remove chlorophenols already present from other sources, and inability to form harmful chloramines and THMs.

ClO₂ is also anticipated to use with materials that should not be exposed to high relative humidity/moisture. For example, the present method is contemplated for use with cosmetics, cosmetic packaging, tattoo needles, artifacts, books, artwork, cannabis and tabaco products.

The exemplary embodiments have been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

To aid the Patent Office and any readers of this applications and any resulting patent in interpreting the claims appended hereto, applicants do not intend any of the appended claims or claim elements to invoke 35 U.S.C. 112(f) unless the words “means for” or “step for” are explicitly used in the particular claim.

1. A sterilization apparatus configured for reducing microbial content of an article, the apparatus comprising: (i) a chlorine dioxide generation system for generating chlorine dioxide gas, (ii) a treatment chamber configured to receive the article, said treatment chamber being pressurized between 2 and 120 kPa, with an atmosphere that can have a relative humidity from about 20% to about 100% at a temperature from about 2 degrees Celsius to about 80 degrees Celsius, (iii) a ClO₂ scrubber, and (iv) and one or more sensing unit(s) in combination with a controller having software configured to sense and optionally adjust one or more conditions in the chlorine dioxide generation system, treatment chamber, and/or ClO₂ scrubber.

2. The apparatus of claim 1 including a buffer tank receiving the chlorine gas and building a pressure of at least 100 kPa.

3. The apparatus of claim 1 including an evacuation tank for receiving the chlorine dioxide gas after exposure to the article, said evacuation tank including a degradation enhancement device.

4. The apparatus of claim 1 wherein the chlorine dioxide is pulsed from the chlorine dioxide generation system into the treatment chamber.

5. The apparatus of claim 3 including a recirculation of chlorine dioxide from the evacuation tank to the treatment chamber.

6. The apparatus of claim 1 wherein the treatment chamber includes a means of aeration after sterilization of the article.

7. The apparatus of claim 3 wherein the degradation enhancement device comprises a UV lamp and//or a microwave generator.

8. The apparatus of claim 2 further including a steam generator for increasing relative humidity in the treatment chamber.

9. The apparatus of claim 8 wherein at least two of the ClO2 generation system, treatment chamber, steam generator, and ClO2 scrubber include a temperature detector.

10. The apparatus of claim 8 wherein at least two of the ClO2 generation system, treatment chamber, steam generator, and ClO₂ scrubber include a relative humidity detector.

11. The apparatus of claim 8 wherein at least two of the ClO2 generation system, treatment chamber, steam generator, and ClO2 scrubber include a pressure detector.

12. The apparatus of claim 8 wherein at least two of the ClO2 generation system, treatment chamber, evacuation tank, and ClO2 scrubber and include a gas concentration detector.

13. The apparatus of claim 8 wherein at least two of the ClO2 generation system, treatment chamber, steam generator, and ClO2 scrubber include a time detector.

14. The apparatus of claim 8 wherein the steam generator increases the temperature of the ClO₂ to at least 2° Celsius in the treatment chamber.

15. (canceled)

16. The apparatus of claim 1 wherein at least one of the treatment chamber and/or ClO₂ scrubber further includes a magnetic field generator.

17. (canceled)

18. (canceled)

19. The apparatus of claim 1 wherein the treatment chamber is capable of receiving a pallet of articles comprising a volume of at least 200 liters.

20. The apparatus of claim 1 wherein the chlorine dioxide generation system is electrochemical and has a capacity from 0.45 kg/day to at least 136 kg/day ClO₂.

21. The apparatus of claim 1 including a vacuum mechanism for emptying the treatment chamber after sterilization of the article.

22. (canceled)

23. (canceled)

24. (canceled)

25. A sterilization apparatus configured for reducing microbial content of an article comprising a chlorine dioxide electrochemical generation system for generating chlorine dioxide gas from an electrochemical reaction of sodium chlorate (NaClO2) and water (H2O) via an anode oxidation reduction reaction NaClO2→ClO2+e— and a cathode reduction reaction 2H₂O+2e—→H2+20H— yielding an overall scheme in which 2NaClO2+2H₂O→2ClO₂+H₂+2NaOH, said chlorine dioxide gas being provided to a treatment chamber.

26. (canceled)

27. A sterilization method including the steps of arranging an article within a space area, introducing chlorine dioxide gas from an associated system into the space area, sterilizing the article, and introducing the chlorine dioxide after exposure to the article into a stream or volume of water.
